(19) United States
(12) Patent Application Publication
(10) Pub. No.: US 2008/0292779 A1
(43) Pub. Date: Nov. 27, 2008

(54) COATING OF IMPLANTS WITH HYALURONIC ACID SOLUTION

(76) Inventors: Gregory M. Mercuri, Victoria, MN (US); James G. Hall, Woodbury, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/110,641

(22) Filed: Apr. 28, 2008

Related U.S. Application Data
(60) Provisional application No. 60/939,461, filed on May 22, 2007.

(51) Int. Cl. A61L 27/28; A61C 13/00

(52) U.S. Cl. 427/2.26; 427/2.29

(57) ABSTRACT

A method for fabricating and/or coating an implant or other structure to be inserted into bone or osseous tissue, including dental implants, is described in which the surface of the implant is treated with a hyaluronic acid solution to form a thin film conformal coating of the hyaluronic solution over the surface of the implant. The surface of the implant may be roughened and cleaned prior to treating the surface with the hyaluronic solution. After the coating is formed, the implant may be sterilized.

### RELATED APPLICATIONS

The present application claims the benefit of U.S. Provisional Patent Application No. 60/939,461, entitled “Coating of Implants with Hyaluronic Acid Solution,” filed on May 22, 2007, the contents of which is incorporated herein by reference.

### BACKGROUND OF THE INVENTION

The present invention relates generally to the field of implants that are installed in bone or osseous tissue and more specifically to dental implants. The invention also relates to a method of fabricating or coating such implants.

Dental implants commonly include a body with external threads for mounting and retaining the implant within the jawbone of the patient. Installation of the implant involves rotation of the implant into a predrilled or tapped site using a drive member such as a ratchet or other rotation means. Over time, the implant is integrated into the bone, a process referred to as osseointegration.

The osseointegration of a dental implant may take a significant period of time, for example, twelve or more weeks. Upon initial installation of an implant, the implant may be screwed into place such that the screw threads on the implant hold the implant secure for the first few weeks. As the surgical site goes through the healing and remodeling process, the bone that was disturbed during surgery is removed and replaced by fresh bone. This process may cause the dental implant to temporarily become less stable than at insertion. The healing and remodeling process typically takes several months, thus requiring a waiting period before restoring an implant. In patients with compromised biology, this waiting period may be much longer. However long, during the waiting period the patient does not have functioning teeth.

Therefore, there is a need for an implant such as a dental implant with an improved surface characteristic that allow for faster osseointegration of implants, achieving benefits such as shortening the waiting period before patients are ready to receive restorations.

### SUMMARY OF THE INVENTION

The present invention relates to a method for fabricating and/or coating an implant or other structure to be inserted into bone or osseous tissue, including dental implants, in which the surface of the implant is treated with a hyaluronic acid (HA) solution to form a thin film conformal coating of HA over the surface of the implant. This process increases the hydrophilic nature of the implant surface such that the growth factors and proteins necessary for osseointegration are more readily attracted to the implant surface and increase the rate at which the bone heals. As a result, a patient may receive a restoration more quickly. The reliability of implants in compromised surgical sites may also be improved.

A method for fabricating an implant may include: machining the implant; roughening the implant surface; cleaning the implant surface; coating the cleaned implant surface with a hyaluronic acid solution to deposit a thin film conformal coating of HA over the surface of the implant; drying the coating; and sterilizing the implant.

A method for coating an implant may include: roughening the surface of an implant; cleaning the implant surface; coating the cleaned implant surface with a hyaluronic acid solution to deposit a thin film conformal coating of HA over the surface of the implant; and drying the coating.

These and other features and advantages of the present invention will become apparent to those skilled in the art from the following detailed description, wherein it is shown and described illustrative embodiments of the invention, including best modes contemplated for carrying out the invention. As it will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 provides a flow chart of an exemplary method for fabricating implants.

### DETAILED DESCRIPTION

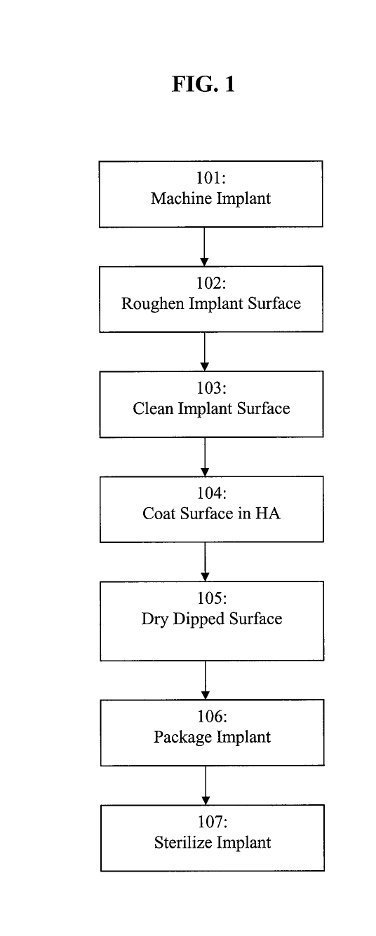

With reference to FIG. 1, a method for fabricating an implant or other structure to be inserted or otherwise positioned in bone or osseous tissue may include the following:

101—Machine an implant, such as a dental or other implant or structure to be implanted or otherwise positioned in bone or osseous tissue. For example, the implant may be fabricated using titanium or any other desired material.

102—Roughen the surface of the implant. For example, a grit blasting procedure using a calcium phosphate media or any other grit blasting media may be used to achieve a roughened surface on a titanium implant. Alternatively or additionally, acid etching, implemented alone or subsequent to the grit blasting procedure, or other roughening process may be used to roughen the implant surface. Roughening is performed on at least a portion of the implant surface that is to be positioned in contact with the bone or osseous tissue.

In an alternative embodiment, the roughening process may be omitted. Thus, the coating process described below may be performed on any implant having a roughened or smooth surface or combination of surface textures.

103—Clean the roughened surface, for example, using a plasma cleaning process performed in an inert atmosphere such as argon. The cleaning process removes all surface contaminants and creates an atomically clean surface. When a titanium implant is used, the cleaning process also increases hydrophilicity of the titanium surface. During the cleaning process, the implant is positioned such that the plasma cleaning does not alter the critical dimensions or chemistry of the internal features of the implant.

If plasma cleaning is utilized, any inert gas (such as argon) may be used in the plasma cleaning process, for example, over an energy range of 500 eV to 1.5 keV. Radio frequency (RF) or direct current (DC) plasma cleaning processes may be used. DC plasma also may be used in conjunction with ion beam sputtering to create a microtextured surface overlayed on the roughened surface of the implant.

Also, when plasma cleaning is used, plasma modification of the implant surface allows for chemical attachment of the hyaluronic acid solution to the implant surface as described below.

104—Coat the cleaned implant with a hyaluronic acid (HA) solution (a biocompatible solution) to create a conformal thin-film coating of HA over the surface of the implant. Coating may be accomplished by dipping, spraying, or any other desired coating process. In one example in which a dipping process is used to create the HA coating, HA may, for example, be dissolved in water or a buffered solution to create a dipping solution. The molecular weight of the HA solution may have any molecular weight as needed to provide the desired HA coating. For example, an HA solution having an average molecular weight of 5,000-2,500,000 Daltons may be used. However, a wide range of molecular weights and formulations of HA may be employed. The pH of the HA coating may range from approximately 6.0 to approximately 6.9. For example, an HA solution having a pH of approximately 6.4 may be utilized. Additional details concerning HA are provided below.

105—After the implant is dipped in the HA solution, dry the implant. For example, an air drying process, a heating process, or any other desired drying process may be used.

106—After the implant is dry, the implant optionally may be packaged.

107—After packaging, the implant optionally may be sterilized, for example, Gamma sterilized with a dose of 25-40 kGy. Gamma sterilization produces a specific molecular structure of the HA that may increase the hydrophilic properties of the surface. In particular, irradiation produces a molecular structure of HA having a molecular weight of approximately 35,000-45,000 Daltons.

Alternatively, any other desired sterilization process, such as using ethylene oxide, may be used.

The HA surface of the implant that is created using the method described above with reference to FIG. 1 may be used as a medium for bioactive compounds such as bone morphogenic proteins (BMP), specifically BMP2, BMP3, and BMP7. Other growth factors, peptides, or drugs such as corticosteroids may attach to the bioactive compounds.

Hyaluronic acid (HA) used to coat the implant in the process described above with reference to FIG. 1 is a glycosaminoglycan, also called a mucopolysaccharide. This polysaccharide consists of repeating units of disaccharides of alternating D-glucuronic acid and N-acetylglucosamine molecules. These molecules are joined by a β (1,3)-D linkage while the glucosamine to glucuronic acid linkage is β (1,4) D.

Many uses for HA are possible as it is hydrophilic and can act as a humectant or lubricant in products. HA is utilized as an ingredient in many familiar products such as cosmetic formulations, including skin care products, injectable line and wrinkle fillers, food additives, dietary supplements, pharmaceutical uses and products for other medical uses such as joint fluid replacement. The sodium salt, sodium hyaluronate has been used as an ingredient in eye vitreous replacements in optical surgery or in eye drops.

HA can be extracted from animal derived sources such as rooster combs, human umbilical cords, bovine (cows, oxen) body parts or other vertebrate tissue including skin tissue, joint fluids, vitreous humor of the eye and cartilage. HA can also be produced using biotechnological methods utilizing bacterial sources including strains of Streptococcus bacteria, Bacillus bacteria and recombinant bacteria. HA has the same chemical composition no matter the source.

HA often plays an important role in the biological organism, firstly as a mechanical support of the cells of many tissues, such as the skin, the tendons, the muscles and cartilage and it is therefore the main component of the extracellular matrix. But HA also performs other functions in the biological processes, such as the hydration of tissues, lubrication, cellular migration, cell function and differentiation.

The term hyaluronic acid or HA is known in the art and it should be understood, that the term “hyaluronic acid” includes hyaluronan. Hyaluronic acid, under physiological conditions, is converted into various forms, based on electrolytes and other physiological medium. Therefore, it should be understood that once the hyaluronic acid is placed in an electrolytic solution, it is more correctly known as hyaluronan.

The method described above with reference to FIG. 1 may provide a number of benefits, including one or more of the following. Roughening the surface of the implant (102 in FIG. 1) creates a surface texture that allows for more bone-to-implant surface contact. This surface texture also allows for ingrowth of bone into the implant surface, which results in osseointegration of the implant into the bone. This osseointegration keeps the implant stable for the long term.

The cleaning process (103 in FIG. 1) removes all contaminants from the implant surface and, in the case of titanium and other metal implants, increases the surface free energy of the implant. Increasing the surface free energy of an implant surface increases the ability of the surface to attract liquids and other hydrophilic substances such as HA. In particular, by cleaning the implant surface prior to coating the implant with an HA solution, an improved bond is formed between the implant surface and the HA coating. Once formed, the HA coating increases the hydrophilic properties of the coated implant surface and also preserves the increased surface free energy of the implant surface under the coating. Thus, as the HA coating breaks down after the implant is inserted into the bone, the preserved implant surface is exposed, increasing the ability of the preserved surface to attract liquids and other hydrophilic substances and thereby enhancing osseointegration of the implant.

By coating the implant in HA after cleaning (104 in FIG. 1), the base metal (e.g., titanium) is protected and its surface free energy is maintained by the HA coating. HA is also very hydrophilic in the dried state. Therefore, an implant coated with HA, when implanted, attracts blood and the bone growth factors more rapidly than a standard implant without HA.

HA is also believed to have angiogenic properties. Angiogenesis is the process by which new blood vessels grow from current blood vessels. Thus, the HA coating may encourage new blood vessel growth around the implant, expediting the healing process.

The HA coating may also produce increased cell density on the surface of the implant. For example, in one laboratory experiment, six tissue culture experiments were conducted to assess differences, if any, in the way that bone precursor cells (osteoblasts) respond to an HA coated surface versus a resorbable blast medium (RBM) treated surface. Donor normal human osteoblasts were utilized for the testing. The initial testing was performed to establish the suitability of the test method. The test methods and procedures were determined to be scientifically sound and these procedures were used to conduct the final feasibility testing described below.

The final feasibility experiments were designed to look at early time points in terms of cell adhesion. 12 RBM treated disks and 12 RBM treated/HA coated disks (1% solution in WFI, powder lot #P9805-9A) were placed in a tissue culture medium and osteoblasts seeded on each disk. The cells attached to each disk were subsequently counted on two disks each at 4, 6 and 8 hours post seeding. At four hours, there were 29% more osteoblasts on the HA coated surface than on the RBM treated surface, at 6 hours, 11% more and at 8 hours, 32% more. This data indicates that donor human osteoblast cells may respond more readily to HA coated surfaces than to an RBM treated surface. The data suggests an HA coated surface is better able to retain osteoblast cells compared to an RBM surface.

From the above description and drawings, it will be understood by those of ordinary skill in the art that the particular embodiments shown and described are for purposes of illustration only and are not intended to limit the scope of the present invention. Those of ordinary skill in the art will recognize that the present invention may be embodied in other specific forms without departing from its spirit or essential characteristics. References to details of particular embodiments are not intended to limit the scope of the invention.

We claim:

1. A method of fabricating an implant, comprising:
machining an implant;
cleaning the surface of the implant;
coating the cleaned implant surface using a solution of hyaluronic acid to create a conformal layer of the hyaluronic acid solution over the surface of the implant; and
drying the dipped implant surface.

2. The method of claim 1, wherein the implant is a dental implant.

3. The method of claim 1, wherein the implant is fabricated from titanium.

4. The method of claim 1, wherein the implant is cleaned using plasma cleaning.

5. The method of claim 4, wherein the plasma cleaning is performed in an inert atmosphere.

6. The method of claim 1, further comprising:
roughening the surface of the implant prior to cleaning the implant.

7. The method of claim 1, further comprising:
after drying the implant, sterilizing the implant using Gamma radiation.

8. The method of claim 1, wherein the coating is formed by dipping the implant into the hyaluronic acid solution.

9. A method of coating a dental implant, comprising:
roughening the surface of a dental implant;
cleaning the roughened surface of the implant using plasma cleaning;
coating the cleaned implant surface using a solution of hyaluronic acid to create a conformal layer of the hyaluronic acid solution over the surface of the implant;
drying the dipped implant surface; and
sterilizing the implant.

10. A method of coating a titanium implant, comprising:
roughening the surface of a titanium implant;
cleaning the roughened surface of the implant using plasma cleaning to remove an oxide layer from the roughened surface of the implant;
coating the cleaned implant surface using a solution of hyaluronic acid to create a conformal layer of the hyaluronic acid solution over the surface of the implant;
drying the dipped implant surface; and
sterilizing the implant using Gamma radiation.
